(19) United States
(12) Patent Application Publication
(10) Pub. No.: US 2023/0022227 A1
(43) Pub. Date: Jan. 26, 2023

(54) DEVICE AND SYSTEM FOR PASSIVE CLINICAL ASSESSMENT

(71) Applicant: Christiana Care Health System, Inc., Wilmington, DE (US)

(72) Inventor: Kirk Garratt, Wilmington, DE (US)

(21) Appl. No.: 17/944,592

(22) Filed: Sep. 14, 2022

Related U.S. Application Data
(63) Continuation of application No. PCT/US2021/029287, filed on Apr. 27, 2021.
(60) Provisional application No. 63/018,159, filed on Apr. 30, 2020.

(51) Int. Cl. G16H 40/67; G16H 40/40; A61B 5/0205; A61B 5/022; A61B 5/08; A61B 5/308; A61B 5/00

(52) U.S. Cl. CPC G16H 40/67; A61B 5/022; A61B 5/082; A61B 5/308; A61B 5/742; A61B 5/0816; A61B 5/02055; A61B 5/6887; G16H 40/40

(57) ABSTRACT

A device for gathering vital sign and other heath indicator data. The device is constructed to register with a person’s anatomical structure in a predictable fashion, and to include integrated sensors positioned to register with specific anatomical portions of the person. Accordingly, relevant data and assessment may be performed passively, e.g., during physical contact/engagement of the patient with the sensors of the device, which may be for, example, a chair or other piece of furniture in which the patient may sit. The device may analyze gathered sensor data and draw clinical assessment conclusions. Data analysis may be done at the device, or remotely. Vital sign/data and/or assessment conclusions may be displayed to the caregiver only, e.g., via a display device or via an EHR or other computing system receiving data from the device. A separate display screen may display information to the patient for entertainment or instructive purposes.

### CROSS-REFERENCE TO RELATED APPLICATION

This application is a continuation application of International Application No. PCT/US2021/029287, filed Apr. 27, 2021, which claims the benefit of priority, under 35 U.S.C. §119(e), of U.S. Provisional Pat. Application No. 63/018,159, filed Apr. 30, 2020, the entire disclosure of each of which is hereby incorporated herein by reference.

### FIELD OF THE INVENTION

The present invention relates generally to clinical assessment of personal health of patients and other persons as is required for hospital Emergency Department triage and other purposes, and more particularly to a device and system for clinical assessment of persons to gather and/or analyze vital signs and other heath indications in a passive manner that provide a feasible alternative to thorough patient examination by a highly skilled healthcare professional in certain limited circumstances, such as in initial patient screening, intake and/or triage activities.

### DISCUSSION OF RELATED ART

Clinical assessment of patients and other persons to obtain vital signs and other measurable or observable indications of personal health and well-being is necessary or desired in a variety of different environments and contexts. For example, it may be necessary or desirable to measure/assess a person’s heart rate, blood pressure, basal/body temperature, respiratory rate, blood oxygen content, electrocardiogram, weight and/or other vital signs or other health-related indications. By way of example, it may be desirable to obtain such clinical assessment measurements in hospital patient-intake settings, in hospital infusion suites, in physicians' offices, in healthcare providers' offices, in nursing home and long-term care environments, and in medical aid units and in other environments for general monitoring and other purposes.

By way of further example, it is common to obtain such clinical assessment information in the context of patient intake in a hospital’s Emergency Department (ED) setting, for patient triage purposes. Often, as part of patient intake in the ED environment, a nurse or other skilled healthcare professional interviews a person/incoming to gather vital sign and other patient health information to collect information used to determine whether the person needs close medical immediately, or less urgently, so that patients with the greatest needs can be cared for most quickly. For a patient that displays or reports certain symptoms clearly associated with the patient’s medical condition, this can work very well. For example, in accordance with hospital standards, a patient identified initially as having a serious heart problem may be thoroughly examined clinically and receive a determinative diagnostic test and/or care within 10-20 minutes after initial patient contact, which provides for a high likelihood of a successful medical outcome. However, for a patient that does not display or report symptoms clearly associated with the patient’s medical condition, this approach can work less well, leading to mischaracterization of the urgency of the patient’s need for care. For example, such an atypical presentation of a heart disorder is not uncommon among women, who may present with atypical complaints associated with a heart attack or other heart problems. In absence of typical presenting symptoms, if the patient is not identified initially as having a serious heart problem, delays in obtaining vital information and determinative testing and/or care are likely to occur, thereby decreasing the likelihood of a successful medical outcome. In such a case, an initial mischaracterization of the patient’s need for care as other than urgent may result in a heart attack, or patient death.

The patient’s medical condition and/or need for care is often more clearly definable after a clinical assessment that involves a thorough patient examination and clinical assessment by a health care professional. However, such a clinical assessment is routinely performed in a private or semi-private area (typically not in the patient intake area of the ED) and by one or more highly skilled healthcare professionals, using specialized equipment. Because the availability of private/semi-private areas, skilled healthcare professionals, and the available time of such professionals is limited, triage is necessary, and it is important to triage patients accurately prior to admission to a private/semi-private area and/or prior to a thorough clinical examination by a skilled healthcare professional. Collection of vital information at an earlier point in the episode of care would enhance healthcare efficiencies and reduce risk of avoidable adverse outcomes.

Although various devices exist for checking various patient vital signs, etc., a skilled healthcare professional is often needed for accurate measurements. Additionally, consumer-grade devices typically require effort by the patient and are prone to user error leading to inaccurate measurements.

What is needed is a device that provides for clinical assessment of persons to gather and/or analyze vital signs and other heath indicators prior to thorough patient examination by a highly-skilled healthcare professional, e.g., such as in initial patient screening, intake and/or triage activities, to provide a feasible alternative to thorough patient examination, and to reduce delays in thorough patient examination, and ease the demand for limited resources, such as private/semi-private patient examination areas, skilled healthcare professionals, and the available time of such professionals.

### SUMMARY

The present invention provides a device that provides for clinical assessment of persons to gather and/or analyze vital signs and other heath indicators prior to thorough patient examination by a highly-skilled healthcare professional. The device and system thereby provides a feasible alternative to thorough patient examination, reduces delays in thorough patient examination, and eases the demand for limited resources, such as private/semi-private patient examination areas, skilled healthcare professionals, and the available time of such professionals.

### BRIEF DESCRIPTION OF THE FIGURES

An understanding of the following description will be facilitated by reference to the attached drawings, in which:

FIGS. 1A-1B are perspective views of an exemplary passive clinical assessment device in accordance with an exemplary embodiment of the present invention;

FIG. 2 is a system diagram showing an exemplary network environment in which the present invention may be employed; and

FIG. 3 is a schematic diagram of an exemplary special-purpose Patient Assessment Management System in accordance with an exemplary embodiment of the present invention.

### DETAILED DESCRIPTION

The present invention provides a clinical patient assessment device and system that provide for clinical assessment of persons to gather and/or analyze vital signs and other heath indicators, and as such may be used as a feasible alternative to and/or adjunct to thorough patient examination by a highly-skilled healthcare professional, e.g., such as in initial patient screening, intake and/or triage activities. In this manner, the device and system may be used to support or improve accuracy in triage activities, reduce delays in thorough patient examination, and ease the demand for limited resources, such as private/semi-private patient examination areas, skilled healthcare professionals, and the available time of such professionals. By way of example, the device could be configured as a chair, be disposed in a hospital’s patient check-in area, and be incorporated into intake workflow such that it is used with every incoming patient as part of the routine (non-emergency) check-in process, in a very simple way that would not require a skilled healthcare professional in order to obtain a helpful array of accurate vital sign and/or health indication information. This is advantageous, for example, because it would permit ambulatory patients presenting to the ED to have a traditional EKG sooner, at intake, rather than sometime afterward. For example, it is not uncommon for an initial EKG to be obtained within 10 minutes after arrival among patients suspected of having a heart attack, but a much longer interval may precede obtaining an ECG in a patient presenting with atypical symptoms, in whom heart attack may not be considered. Such delays are known to contribute to poor outcomes Thus, the device would aid in prevention of injury by facilitation early intervention, when needed, and/or optimizing resource utilization.

Accordingly, the device will allow for acquisition of basic vital sign information earlier in the typical patient workflow, as well as facilitate more rapid triage of the patient to an appropriate level of care. Because of this earlier identification, the device will offer particular value in any ED, urgent care center or medical aid unit to help identify patients who may not present with the typical symptoms of a cardiac problem. The device may also have utility in managing/triaging patients with known common cardiac problems, including chronic ischemic heart disease and chronic congestive heart failure, by improving ability to distinguish between cardiac and non-cardiac symptoms. Notwithstanding the foregoing, the device may be used to advantage in any setting in which patient care is provided.

The device in accordance with the present invention integrates various components usable for capturing biometric data of the type relevant to a patient clinical assessment. Further, the device integrates relevant sensors into objects and/or surfaces, such as a countertops, stretchers, tables, and/or chairs, in a strategic fashion, such that the relevant sensors are positioned to readily register with appropriate anatomical structures to ensure accurate measurements without the need for careful attention or effort on the part of the patient and/or the involvement of a skilled healthcare professional. For example, the device’s sensors may be positioned to make contact with an individual’s skin on both upper extremities (hands) and with one lower extremity (leg) to capture and display biometric data relevant to standard electrocardiographic measurement methods, preferably passively or semi-passively. For example, the device’s sensors may be embedded in a section of a chair or similar device in such a manner that they make physical contact with a patient. Contact points can be used to acquire cutaneous galvanic (electrocardiographic) signals and develop and ECG. Sensors can similarly be incorporated into other objects, such as benches, sofas, wheelchairs, gurneys, hospital beds and other places to obtain biometric data intermittently or continuously, and passively, or largely passively.

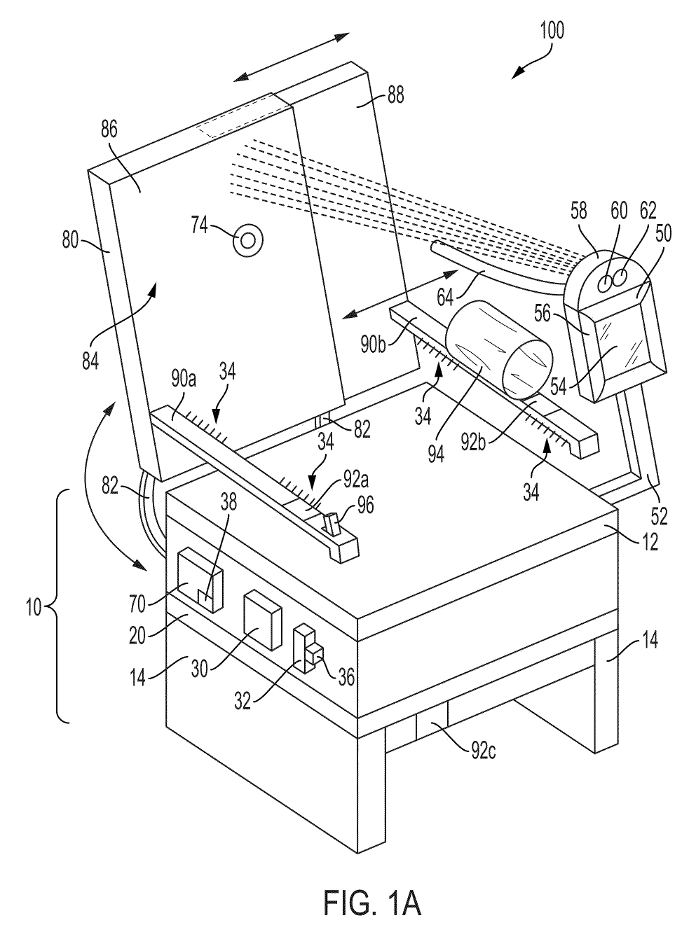

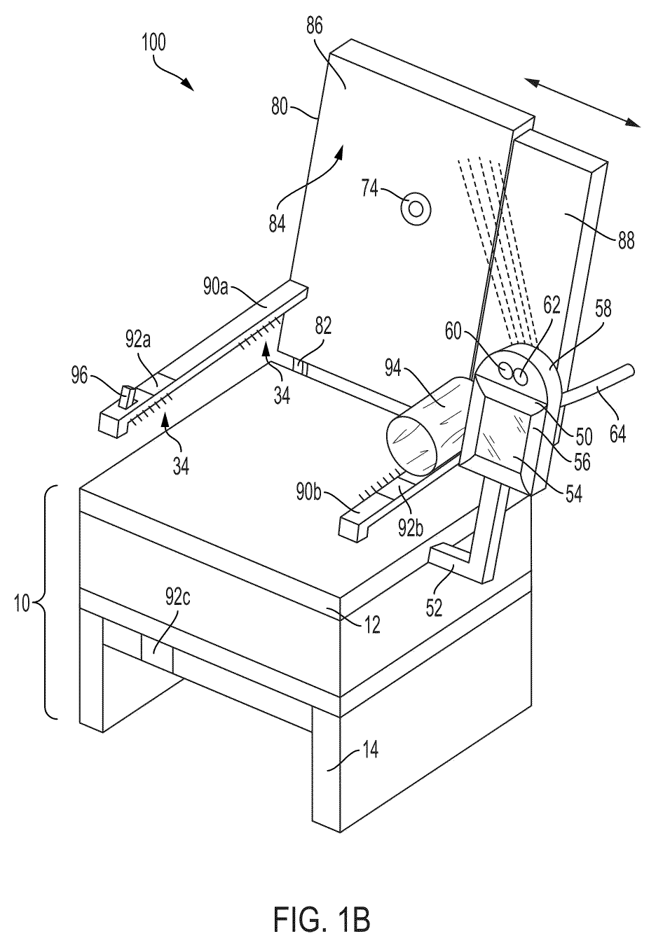

An exemplary embodiment of the present invention is discussed below in reference to an exemplary device 100 configured in the form of a chair for illustrative and non-limiting illustrative purposes only. Referring now to FIGS. 1A-1B, the exemplary clinical patient clinical assessment device 100 is shown. As shown in FIGS. 1A-1B, the device 100 includes a base 10 including a seat 12 and a backrest 80 supported on the base 10. Although the backrest 80 may be fixed relative to the base 10, in this embodiment, the backrest 80 is movable to tilt forwards and backwards relative to the base to allow for a measure of reclining action for the occupant of the device 100. The backrest 80 is joined to the base 10 by a support 82 which may be a fixed structural member that is bendable in the manner of a living hinge, or a mechanical assembly that is manually adjustable to cause the backrest 82 move, or a mechanical assembly that is powered by a motor and drive mechanism to cause the backrest 82 to move.

Alternatively, the seat and backrest, or the entire chair, may be configured to tilt rearwardly, e.g., by about 20 degrees, after the patient has become seated in the chair. In an such case, the movable/tilting features is advantageous in that it helps to relax the patient, helps the patient to assume the right position in the chair for proper alignment of the sensors to the patient’s body (e.g., no slouching), and improves contact of the patient with sensors in the back of the chair.

Consistent with the present invention, multiple sensors are incorporated into the device 100 and appropriately positioned for registering in predetermined fashion with selected portions of the human anatomy to permit obtaining vital sign and/or other biometric and/or health information in a manner that involves little or no action or effort, and no specialized knowledge, on the part of the devices user/occupant, namely, a patient. Accordingly, the base 10 includes legs 14 for supporting the remainder of the device on a ground surface. Additionally, the device 100 includes a scale mechanism 20. By way of brief example, the scale mechanism 20 may include an analog scale including springs etc. disposed between the seat 12 and the legs 14. By way of alternative example, the scale mechanism 20 may include a digital scale including strain gauges attached to load cells disposed between the seat 12 in the legs 14. The scale mechanism 20 may have a suitable structure, and various structures are known in the art for use for this purpose, and thus are not discussed in detail herein. Scale mechanism is usable to obtain a measurement of the weight of a person seated in the chair device 100. In a preferred embodiment, scale mechanism 24 is provided as a digital scale that is capable of communicating weight data to an electronic/computerized data management component (DMC) 70 of the device 100. Hardware and software for developing a weight measurement from such data are well-known in the art. The scale mechanism 24 is operatively connected to the DMC 70 to communicate data to the DMC 70, and that data may be processed at the DMC 70 or elsewhere to develop a weight measurement relevant to a clinical assessment of the patient.

The backrest 80 includes a sensor 74, such as a microphone, supported on an inner surface 84 of the backrest 80. The sensor 74 is positioned in a location to register with a back portion of a person seated in the chair device 100, adjacent the patient’s heart and/or lungs, so as to be appropriate for auscultation. In one embodiment, the sensor 74 may include multiple microphones, e.g., one positioned for alignment with the left lung, and another positioned for alignment with the right lung, so that lungs may be auscultated separately. The microphone/sensor 74 captures an audio/other signal of the patient’s breathing and gathers data that may be used by the DMC 70, for example to measure the person’s respiratory rate, and/or to complement EKG findings about heart rhythm, murmurs, etc., e.g., to draw conclusions or issue alerts based on a comparison or captured audio signals to prestored digital waveforms associated with potentially worrisome murmurs. Hardware and software for doing so are well-known in the art. The sensor 74 is operatively connected to the DMC 70 to communicate data to the DMC 70, and that data may be processed at the DMC 70 or elsewhere to develop a respiratory rate measurement or other conclusion relevant to a clinical assessment of the patient.

In this exemplary embodiment, the backrest is width-adjustable, to accommodate a greater range of body sizes. This embodiment the backrest 80 includes a fixed portion 86 that is joined to the base 10 and a movable portion 88 that is movable relative to the fixed portion 86. In this example, the movable portion 88 is movable laterally to extend or retract the movable portion 88 relative to the fixed portion 86. In this embodiment, the retracted portion of the movable portion 88 nests within the fixed portion 86. In alternative embodiments the movable portion 88 may move in front of or behind the fixed portion 86. Any suitable mechanism may be employed to allow for the movable portion to move, and the mechanism may be manually adjustable or may be electrically powered by an electric motor. By way of example, a "rolling desktop"-type system that retracts a portion of the right side and an equivalent portion of the left side of the backrest simultaneously to narrow the chair, may be used. This arrangement helps to ensure that the patient is located centrally with respect to sensors, especially those on the backrest, of the chair.

Further, the device 100 includes armrests 90a, 90b. In this embodiment, the armrests are joined to and supported on the backrest 80, but in other embodiments, one or more of the armrests may be joined to and supported on the base 10. In either case, the armrests 90a, 90b are provided in positions for supporting the occupant’s arms in a comfortable fashion. In embodiments in which the backrest is not width-adjustable, one or both of the armrests 90a, 90b may be movable to adjust the distance between the armrests to accommodate a greater range of body sizes. By way of example, armrests 90b may be joined to the base 10 rather than to the backrest 80, and may be supported with a mechanism that allows for lateral adjustment of the armrest 90b.

In any case, each of the arms 90a, 90b includes a respective set of conductive contacts 92a, 92b (e.g., for use as positive and negative terminals) positioned to register with the occupant’s wrists or hands and the patient is seated in the chair device 100. Additionally, a third set of conductive contacts 92c is provided on the base 10 of the chair, e.g., between the legs 14. This set of conductive contacts 92c is positioned to register with skin on one of the patient’s legs, which the occupant is seated in the chair device (e.g., after lower a sock or rolling up a pant leg). Accordingly, these three sets of conductive contacts provide appropriately positioned electrodes for a 6-lead electrocardiogram (EKG) using the patient’s left arm, right arm and leg. This combination, unlike many simpler consumer devices, will provide a clinically meaningful and reliable EKG information suitable for clinical assessment, for reasons that will be appreciated by those skilled in the art. For example, the EKG may capture waveform information usable to determine the likelihood of an acute heart problem, including but not limited to acute heart attack requiring treatment without delay. This information could be obtained in all measured patients without incremental cost. These are critical measures for triage purposes. Hardware and software for performing automated interpretation of EKG data are known in the art. These conductive contacts 92a, 92b, 92c are operatively connected to the DMC 70 for EKG analysis and/or data recording/sharing purposes. The gathered data may be processed at the DMC 70 or elsewhere to develop an EKG interpretation relevant to a clinical assessment of the patient.

Further, one of the arms, arm 90b in this exemplary embodiment, includes an inflatable cuff 94 and associated hardware and/or software (which may be incorporated into the DMC 70) for automatedly obtaining a blood pressure measurement of a person seated in the chair device 100 with their forearm passing through inflatable cuff 94. Various cuff, and associated hardware and software for doing so are well-known in the art. The cuff 94 and/or associated hardware and/or software are operatively connected to the DMC 70 to communicate blood pressure data to the DMC 70, and that data may be processed at the DMC 70 or elsewhere to develop a blood pressure measurement relevant to a clinical assessment of the patient. Alternatively, a sensor for a cuff less blood pressure measurement may be used in substitution for the inflatable cuff. Hardware and software for cuffless blood pressure measurements are well known in the art.

Additionally, the chair device 100 includes a pulse oximeter for obtaining a measurement of a level of oxygenation of a person’s blood. In this embodiment, a pulse oximeter clip 96 is fixed to one of the arms, in this example arm 92a. The clip 96 is positioned on the arm 92 a in a position to register with a fingertip of a person seated in the chair device 100. The clip 96 is configured with light sources and photosensors for obtaining an oxygenation level measurement using cutaneous oximetric techniques as is well-known in the art. Hardware and software for doing so are well-known in the art. The clip 96 is operatively connected to the DMC 70 to communicate data to the DMC 70, and that data may be processed at the DMC 70 or elsewhere to develop an oxygenation level measurement relevant to a clinical assessment of the patient.

Additionally, the clip 96 may include additional light sources or other sensors for capturing data usable to determine changes in heart rate, arrhythmias, etc. Technologies for estimating advanced cardiodynamic parameters, including but not limited to cardiac stroke volume, cardiac output, and cardiac index based on photoplethysmographic measures are well-known in the art. Hardware and software for doing so are well-known in the art. These advanced cardiac measures cannot currently be obtained in any other non-invasive manner, but rather requires invasive monitoring involving substantial risk, discomfort and cost to patients. The clip 96 is operatively connected to the DMC 70 to communicate measurement data to the DMC 70, and that data may be processed at the DMC 70 or elsewhere to develop measurements relevant to a clinical assessment of the patient including data used for such advanced cardiodynamic measurement purposes.

The chair device 100 further includes a display pod 50, which in this exemplary embodiment is mounted on an arm 52 extending from the base 10. The display pod 50 includes a caregiver display device 54 that is positioned to be viewable from a vantage point opposite and occupant of the chair device 100, for example, by a nurse standing in front of a patient seated in the chair or other hospital personnel seated and intake/patient registration desk opposite the chair device 100. This caregiver display device 54 may be oriented such that the display device, and any information displayed thereon, is not viewable by the person while seated in the chair device 100. The display pod 50 may include a cowl 56 surrounding the caregiver display device 54 to further obscure the caregiver display device 54 from the chair occupant’s view while also making it viewable to a person/caregiver seated or standing across from the chair’s occupant. The caregiver display device 54 may be any suitable display device, such as an LCD display or an LED display similar to that of a computer display monitor. The caregiver display device 54 is operatively connected with the DCM 70 for displaying information relating to data gathered by the various sensors of the chair device 100. By way of example, the display 54 may be caused by the DCM to display weight, blood pressure, temperature, respiratory rate, heart rate, EKG graphs and/or other data based on data gathered by the sensors of the chair device 100. By way of additional example, the display 54 may be caused by the DCM 70 to display interpretations/analyses/conclusions based on data gathered by the sensors. For example, the display may display textual information messages supporting the triage and/or clinical assessment process such as ADMIT - HIGH PRIORITY, ADMIT - LOW PRIORITY, URGENT CARE REQUIRED, HIGH RISK, MODERATE RISK, LOW RISK. By way of alternative example, the display may display atextual information messages supporting the triage and/or clinical assessment process, such as a red-colored graphic to indicate a high risk level, a yellow-colored graphic to indicated a moderate risk level, or a green-colored graphic to indicate a low risk level.

Optionally, the display pod 50 of the chair device 100 may further include a patient display device 58. Unlike the caregiver display device 54, the patient display device 58 is positioned to be viewable from a vantage point of an occupant of the chair device 100. The patient display device 58 may be any suitable display device, such as an LCD display or an LED display similar to that of a computer display monitor. The patient display device 58 may be in communication with the DCM 74 and/or a communications network and a remotely-located media content server for displaying messages and/or other media content to the patient. By way of example, the patient display device 58 may be used to display instructive content to the occupant of the chair, to guide the person in use of the chair device, how and when to relax/breathe deeply/pace breathing to facilitate accurate measurements to be taken by the chair device, etc. By way of alternative example, the patient display device 58 may be used to display informational content about a hospital/other facility, upcoming events, health conditions, etc. By way of another alternative example, the patient display device 59 may be used to display entertainment content intended to entertain and/or distract the user, which may be helpful in obtaining accurate measurements by the chair device. By way of another alternative example, the patient display device 59 may be interactive and be used to query the patient regarding signs or symptoms raising concern. Such information could be forwarded to the healthcare team more or less simultaneously with acquired biometric data, permitting assessment of both current vital information and detail pertaining to the patient’s chief medical complaint or concern.

The display for pod 50 may also house additional sensors that do not require direct contact with the skin of the patient, to obtain additional vital sign, biometric and/or other health indication information. In this example, the display pod 50 further includes an infrared (IR) temperature sensor 60 capable of measuring temperature from a distance without direct contact. The IR sensor 60 is positioned to be directed to a region adjacent backrest 80 of the chair device 100 that is likely to be occupied by the forehead of an occupant of the chair. In this manner, the IR sensor 60 effectively registers with the forehead of the occupant of the chair, to obtain a basal/body temperature measurement. Hardware and software for doing so are well-known in the art. The IR sensor 60 is operatively connected to the DMC 70 to communicate measurement data to the DMC 70, and that data may be processed at the DMC 70 or elsewhere to develop a temperature measurement relevant to a clinical assessment of the patient.

In this exemplary embodiment, the display pod 50 further includes a video camera 62 positioned to be directed to a region adjacent to backrest 80 of the chair device 100 that is likely to be occupied by the face/head of an occupant of the chair. In this manner, the video camera 62 effectively registers with the face of the occupant of the chair, to obtain images of the patient that may be interpreted, either by a healthcare provider or by appropriate hardware and/or software of the chair 100 or another device, to discern indications of pain, distress, mental state and/or to perform an emotional assessment. IR sensing of body temperature may also be obtained from other body areas by adjusting the placement of IR sensor 60. Hardware and software for doing so are well-known in the art. The video camera 62 is operatively connected to the DMC 70 to communicate video image data to the DMC 70, and that data may be processed at the DMC 70 or elsewhere to discern indications relevant to a clinical assessment of the patient.

In this exemplary embodiment, the display pod 50 further supports a tube 64 operatively connected to hardware and/or software on the display device for performing an automated breathalyzer analysis to determine certain biochemical measurements such as a blood alcohol level of a person seated in the chair device 100. Hardware and software for doing so are well-known in the art. The tube/breathalyzer hardware/software is operatively connected to the DMC 70 to communicate breathalyzer measurement data to the DMC 70, and that data may be processed at the DMC 70 or elsewhere to discern indications relevant to a clinical assessment of the patient.

The device is preferably constructed of durable materials and components requiring only between-use cleansing with standard hospital cleansing wipes, without the need for disposable components, while also providing a low risk of infection transfer. By way of example, various plastic and vinyl materials commonly used in hospital chairs and beds may be used for this purpose. Additionally, copper and/or silver may be used for the conductive contacts or other portions of the chair because they are not only electrically conductive but also have anti-bacterial properties. Bacteriostatic metallic coatings may be applied over various surfaces features in the device to enhance biometric data collection and reduce any infection risk or concern.

In certain embodiments, the device includes a self-cleaning system. In one such exemplary embodiment, the device 100 includes a reservoir 30 for holding an alcohol-based, bleach-based or other disinfecting solution, a compressed gas source 32, such as a compressed gas cylinder and/or an air compressor, a dispenser structure 34, such as one or more nozzles for producing a mist, in fluid communication with the reservoir and the compressed gas source, a valve 36 for controlling a flow of disinfecting solution and gas from the compressed gas source, and a controller 38 for selectively opening and closing the valve. The controller may be integrated into the DMC 70. Accordingly, after a patient exits the chair device, e.g., as may be sensed by the weight sensor 20, or otherwise in response to a manual or timed input, the controller may cause the valve to open and close to dispense a suitable amount of disinfecting solution onto portions of the chair device to be disinfected, so as to clean the chair device between uses by different persons in an automated fashion, and to reduce cross-contamination risk. In addition, all sensors may be assembled in a configuration other than a chair, including but not limited to benches, sofas, gurneys, and hospital beds. Furthermore, a collection of sensors may be produced that can collect some or all intended biometric data from a standing position, for example, a countertop sensor array. Cleansing components may be scaled to suit the requirements of each environmental application.

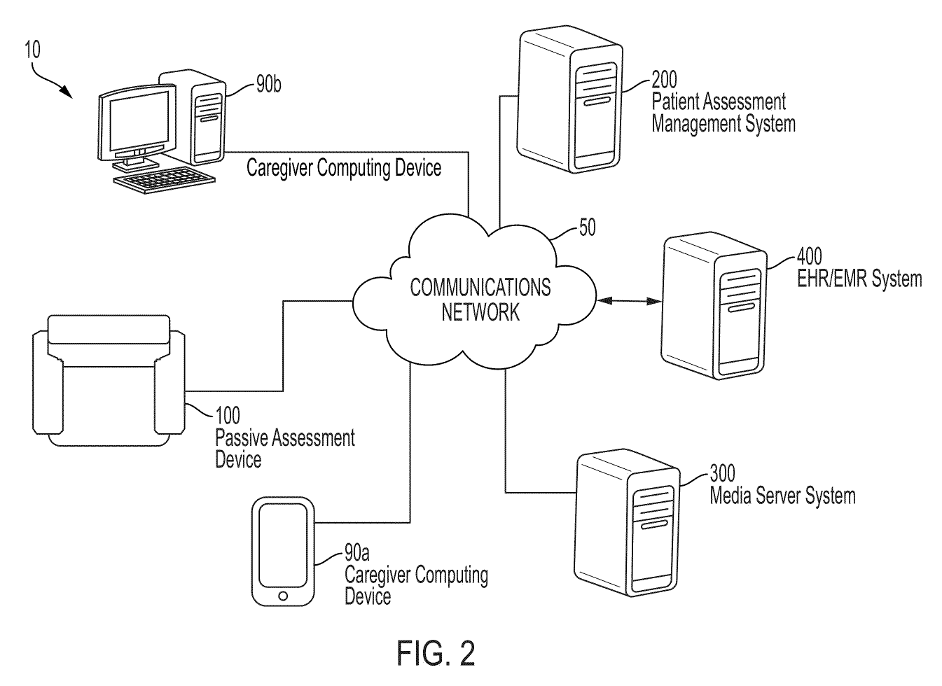

FIG. 2 is a system diagram showing an exemplary network environment in which the present invention may be employed. As shown in FIG. 2, the exemplary network environment 10 includes conventional computing hardware and software for communicating via a communications network 50, such as the Internet, etc., as the Caregiver Computing Device 90a, 90b (e.g., a personal computer/PC, tablet computer or smartphone). The system also includes an Electronic Health Record/Electronic Medical Record (EHR/EMR) system 200. The EHR/EMR system 200 is operatively connected to the passive assessment device 100 via the communications network, and the passive assessment device 100, via the DMC 70 which may be configured for network data communication, is configured to transmit data to the EHM/EMR system 400 so that it can be entered into the corresponding patient’s medical record/chart, and be stored. By way of example, the DMC 70 may be configured with, or to interface with, software for automating data integration into a Cerner, Epic, AllScripts or other EHR system. Additionally, the EHR/EMR system 400 is operative connected to the Caregiver Computing Devices 90a, 90b via the communications network 50 so stored data can be viewed. These systems may be existing or otherwise generally conventional systems, at least in part, including conventional software and web server or other hardware and software for communicating via the communications network 50. Consistent with the present invention, these systems may be configured, in conventional fashion, to communicate/transfer data via the communications network 50 in accordance with and for the purposes of the present invention, as discussed in greater detail below. In addition, acquired information may be directed to third-party vendors capable of augmenting patient care by, for example, rendering recommendations about clinical care using artificial intelligence or other means. Integration with the EHR also permits use of acquired biometric information in hospital quality improvement efforts, to improve communication of patient-related data between care teams, to allow monitoring of patient types served by the facilities, and in other activities of value to hospital systems.

Further, in accordance with the present invention, the network environment 10 includes a Media Server system 300 that is operatively connected to the passive assessment device 100 via the communications network 50, and the passive assessment device 100 is configured to display via the patient display device 58 media content intended for patient viewing/consumption that is provided by the Media Server system 300. The Media Server system 300 may be existing or otherwise generally conventional systems, at least in part, including conventional software and web server or other hardware and software for communicating via the communications network 50. Consistent with the present invention, these systems may be configured, in conventional fashion, to communicate/transfer data via the communications network 50 in accordance with and for the purposes of the present invention, as discussed in greater detail below.

Further still, in accordance with the present invention, the network environment 10 includes a Patient Assessment Management System (PAMS) 200 that is operatively connected to the passive assessment device 100 via the communications network 50. In certain embodiments, analysis and interpretation of data gathered by the passive assessment device 100 may be performed at the passive assessment device. In this embodiment, at least some analysis and/or interpretation of data is performed at the PAMS 200. The PAMS may perform such analysis and interpretation, and then communicate information to the patient assessment device to cause appropriate display at the caregiver display device 54, to guide the caregiver in clinical assessment of the patient. Alternatively, the PAMS (or in some embodiments the passive assessment device 100) may communicate data to cause display of information based on data gathered by the sensors and/or analysis/interpretation performed at the PAMS 200 or the passive assessment device 100 to cause display of appropriate information at the Caregiver Computing Device 90a, 90b. Additionally, the PAMS 200 may communicate data based on analysis/interpretation performed at the PAMS 200 to the EHR/EMR system 400 for storage in the corresponding patient’s medical record. The PAMS 200 may include generally conventional hardware and software for communicating via the communications network 50, as well as instructions for configuring the PAMS 200 as a special-purpose machine in accordance with the present invention. Consistent with the present invention, the PAMS 200 may be configured, in conventional fashion, to communicate/transfer data via the communications network 50 in accordance with and for the purposes of the present invention, as discussed in greater detail below.

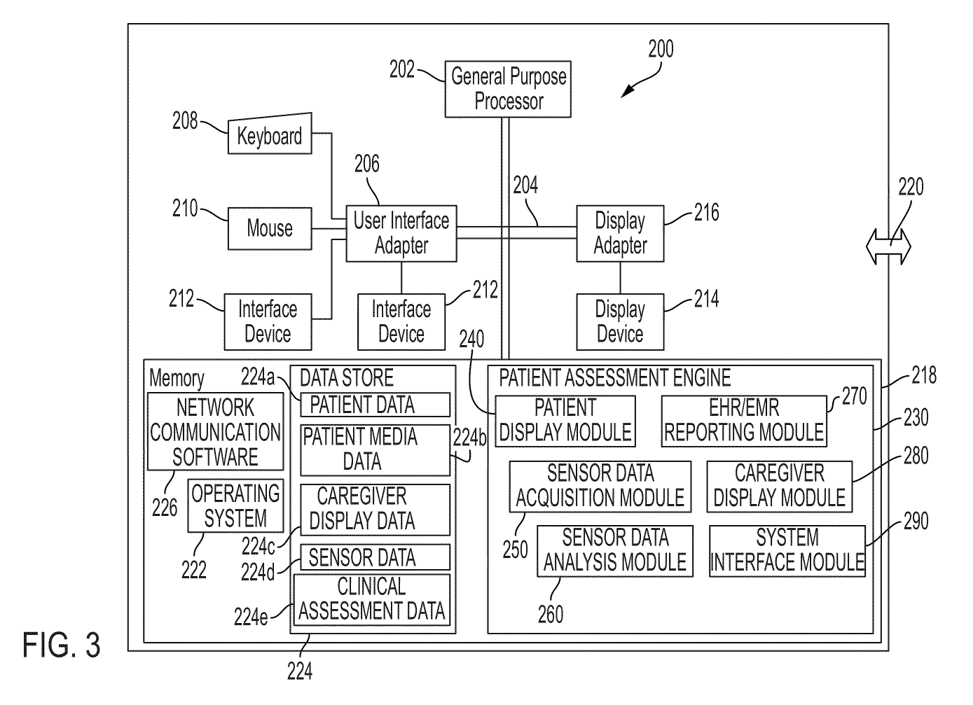

FIG. 3 is a block diagram showing an exemplary Patient Assessment Monitoring System (PAMS) 200 in accordance with an exemplary embodiment of the present invention. The PAMS 200 is a special-purpose computer system that includes conventional computing hardware storing and executing both conventional software enabling operation of a general purpose computing system, such as operating system software 222, network communications software 226, and specially-configured computer software for configuring the general purpose hardware as a special-purpose computer system for carrying out at least one method in accordance with the present invention. By way of example, the communications software 226 may include conventional web server software, and the operating system software 222 may include iOS, Android, Windows, Linux software.

Accordingly, the exemplary PAMS 200 of FIG. 3 includes a general-purpose processor, such as a microprocessor (CPU), 102 and a bus 204 employed to connect and enable communication between the processor 202 and the components of the presentation system in accordance with known techniques. The exemplary presentation system 200 includes a user interface adapter 206, which connects the processor 202 via the bus 204 to one or more interface devices, such as a keyboard 208, mouse 210, and/or other interface devices 212, which can be any user interface device, such as a touch sensitive screen, digitized entry pad, etc. The bus 204 also connects a display device 214, such as an LCD screen or monitor, to the processor 202 via a display adapter 216. The bus 204 also connects the processor 202 to memory 218, which can include a hard drive, diskette drive, tape drive, etc.

The PAMS 200 may communicate with other computers or networks of computers, for example via a communications channel, network card or modem 220. The PAMS 200 may be associated with such other computers in a local area network (LAN) or a wide area network (WAN), and may operate as a server in a client/server arrangement with another computer, etc. Such configurations, as well as the appropriate communications hardware and software, are known in the art.

The PAMS 200 is specially-configured in accordance with the present invention. Accordingly, as shown in FIG. 3, the PAMS 200 includes computer-readable, processor-executable instructions stored in the memory 218 for carrying out the methods described herein. Further, the memory 218 stores certain data, e.g. in one or more databases or other data stores 224 shown logically in FIG. 3 for illustrative purposes, without regard to any particular embodiment in one or more hardware or software components.

Further, as will be noted from FIG. 3, the PAMS 200 includes, in accordance with the present invention, a Patient Assessment Engine (PAE) 230, shown schematically as stored in the memory 218, which includes a number of additional modules providing functionality in accordance with the present invention, as discussed in greater detail below. These modules may be implemented primarily by specially-configured software including microprocessor-executable instructions stored in the memory 218 of the PAMS 200. Optionally, other software may be stored in the memory 218 and and/or other data may be stored in the data store 224 or memory 218.

Referring now to FIGS. 1A, 1B, 2 and 3, in an exemplary embodiment, the PAE 230 or the PAMS 200 controls operation of the passive assessment device (PAD) 100 and performs certain functions. In another embodiment one or more of the functions of the PAMS 200 are performed at the PAD 100, e.g., at least in part by the DMC 70, and thus the DMC 70 and/or the PAD 100 includes the engine and modules, and stores the data, described here with reference to the PAMS 200. In another embodiment, all of the functions of the PAMS 200 are performed at the PAD 100, e.g., at least in part by the DMC 70, and thus the DMC 70 and/or the PAD 100 includes the engine and modules, and stores the data, described here with reference to the PAMS 200. For illustrative purposes, operation of the system is described below with reference to performance of the functions at the PAMS, although they may be performed in whole or in part by the PAD 100.

In use, the PAD 100 may be deployed into a patient intake area of a hospital, e.g., across a desk from a hospital’s intake personnel. An incoming patient may sit comfortably in the PAD 100 during the intake process. The PAD may store and retrieve patient data 224a from the data store 224 of the PAD 100. By way of example, the patient data may be received via a Caregiver Computing Device 90a, 90b in communication with the PAD 100 and/or by input of information at the PAD 100 and/or by retrieval of information from an EHR/EMR System 400 by communication via the communications network 50.

The Patient Display Module 240 of the Patient Assessment Engine 230 may cause display of media content, such as instructions, information and/or entertainment content, to the chair’s occupant, via the patient display device 58. This may involve the PAMS 200 communicating with an external Media Server system 300 via the network 50 to retrieve suitable content and/or may involve retrieval of patient media data 224b from the data store 224 of the PAMS 200. For example, the patient display device 58 may display instructions guiding the patient to sit back in the chair device 100, with hands wrists or arms against the contacts 92a, 92b of the arms 90a, 90b, with a forearm extended through the blood pressure cuff 94, with a finger engaged in the clip 96, and with the skin of one leg abutting contact 92c, to relax, to breathe deeply and/or in a certain cadence, to blow into a breathalyzer tube 64, etc., and this may be done in stages in synchronization with information displayed via the patient display device 58.

The Sensor Data Acquisition Module 250 of the Patient Assessment Engine (PAE) 230 then works to gather data via each of the sensors of the chair device, which may be done in stages in synchronization with information displayed via the patient display device. Accordingly, for example, sensor data may be gathered from/via sensor/microphone 74, contacts 92a, 92b, 92c, clip 96, cuff 94, tube 64, weight sensor 20, IR sensor 60 and/or videocamera 62. Data gathered via the sensors by the PAE 230 may be stored in a corresponding patient’s record in as Patient Data 224a and/or as Sensor Data 224d in the data store 224.

As relevant data is gathered, analysis and/or interpretation of the gathered data may be performed by the Sensor Data Analysis (SDA) module 260. For example, the SDA 260 may interpret an electrical signal/data from a sensor, such as a load cell of the weight sensor 20 to create a measurement of the weight of the occupant of the chair, or may create a blood pressure measurement based on data gathered via the cuff 94. By way of additional example, analysis and/or interpretation of the data may involve further analysis, such as “reading” (interpreting) an EKG created from data gathered from sensors 92a, 92b, 92c, and determining a risk level, or an observation, or a recommendation, or a diagnostic conclusion. Hardware and software for doing so are well-known in the art. The results of any such interpretation and analysis may be stored in a corresponding patient’s record in as Patient Data 224a and/or as Clinical Assessment Data 224e in the data store 224. Additionally, such information may be communicated to the EHR/EMR System 400 for storage in a memory of the system, in association with the corresponding patient’s medical record, so that it may be subsequently accessed, e.g., via a Caregiver Computing Device 90b, 90a. The reporting of such data to the EHR/EMR System 400 is performed by the EHR/EMR Reporting Module 270. Acquired data may also be assessed by artificial intelligence or other means to develop clinical decision aids and/or care recommendations both in near-real time and after initial care is rendered; in this manner, appropriate changes in an initial or preliminary care plan can be expedited based on changes in biometric data following initial therapies.

The Caregiver Display Module (CDM) 280 is responsible for providing information/feedback to a caregiver via the passive assessment device 100. For example, if the SDA 260 interprets/analyses the EKG to determine that the chair’s occupant is in a “high risk” category, then the CDM may display an appropriate notification to hospital personnel via the PAD 100, e.g., by display of an appropriate textual or atextual message, color coding, etc. via the caregiver display device 54.

Appropriate notifications/messaging may be determined by the CDM 280, and may use or be based upon Caregiver Display Data 224c stored in the data store 224. Notably, this may be provided to the hospital personnel in blinded fashion, so that the same notification is not plainly visible to the occupant of the PAD 100. As a result, the hospital personnel may be alerted and/or assisted in making an accurate triage decision or other healthcare-related decision, in a passive and unobtrusive fashion that is not disruptive to the patient. Further, because the PAD device 100 is suitable for use by a layperson is essentially passive fashion (simply sitting in the chair or following very simple instructions), and is incorporated as part of the patient intake process in a public/common-area setting prior to overt clinical assessment in a clinical setting, and without the need for other limited staff or facility resources, the patient can be assessed (at least preliminarily) very early in the hospital-patient interaction, to ensure that the patient receives appropriate care as quickly as is appropriate, particularly in view of the needs of other patients needing care within the same timeframe. This assists in proper allocation of limited and precious medical care resources.

The System Interface Module (SIM) 290 has a notification/messaging function somewhat similar to the CCDM 280, but the SIM 290 is responsible for providing suitable notifications/messages to systems other than the PAD 100. Accordingly, for example, the SIM 290 may provide suitable messages/alerts with other systems, such as patient intake management software, that may be accessed on a computer (e.g., Caregiver Computing Device 90b) at a patient intake desk, and may be a system separate from that of the PAD 100. The SIM 290 may interact with other hospital electronic systems, accessed through the communications network, to facilitate communication between care providers and facilitate coordination of care. For example, by interfacing with the EHR, SIM 290 can identify usual care providers (if affiliated with the hospital organization with EHR entries) and deliver a standardized message indicating the patient has visited a care area. Such information exchange is useful in assuring proper care is delivered. SIM 290 may also compare prior information collected about a specific patient and produce notifications of important changes. By way of another example, comparison of newly acquired EKG information may be compared with prior EKGs, and notification of significant interval changes may be made on caregiver display device 54.

Additionally, the SIM 290 may perform higher level tasks and data analysis, and interact with other systems accordingly. For example, the SIM 290, particularly as part of the PAMS 200 rather than an individual PAD 100, may gather information from many individual PADs 100, and look at the patient data in aggregation to identify trends, observations, draw conclusions, etc. across multiple patients. By way of example, observations about patterns of patients seen with elevated temperatures and abnormal respiratory biometric measurements may alert health system officials of the need to reallocate resources to certain areas experiencing higher than expected volumes of patients who may be infected during community outbreaks of virulent respiratory pathogens. By way of another example, “big data”, machine learning and/or artificial intelligence analysis techniques may be used to analyze such data, e.g., to identify trends, infectious disease occurrences, etc. By way of further example, such an analysis may be used to identify common symptoms, and the system may alert staff accordingly (e.g., via a Caregiver Computing Device 90b). Further, such an analysis may identify an expected need for increased staffing or other resources, and the system may alert staff accordingly.

Additionally, computer readable media storing computer readable code for carrying out the method steps identified above is provided. The computer readable media stores code for carrying out subprocesses for carrying out the methods described herein.

A computer program product recorded on a computer readable medium for carrying out the method steps identified herein is provided. The computer program product comprises computer readable means for carrying out the methods described above.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A passive clinical assessment device comprising:
a patient-engaging structure configured to register with a person’s anatomical structure in a predictable fashion;
a plurality of sensors supported on the patient-engaging structure in positions for registering with selected anatomical portions of a person engaged with the patient-engaging structure; and
a caregiver display device for displaying information as a function of data collected by via said plurality sensors.

2. The passive clinical assessment device of claim 1, wherein said patient-engaging structure is a countertop.

3. The passive clinical assessment device of claim 1, wherein said caregiver display is supported on said patient-engaging structure in a position so as not to be viewable by the person while engaged with the patient-engaging structure.

4. The passive clinical assessment device of claim 1, wherein said patient-engaging structure is configured as a patient-supporting structure.

5. The passive clinical assessment device of claim 4, wherein said patient-supporting structure is configured as at least one of a chair, a sofa, a bench, a hospital bed, and a wheelchair.

6. The passive clinical assessment device of claim 4, wherein said patient-supporting structure further comprises:
a base supporting a seat;
a backrest supported adjacent said seat; and
a pair of armrests, each of said pair of armrests being supported on at least one of said base and said backrest, said base, said backrest and said pair of armrests collectively defining structure for supporting a person in a seated position.

7. The passive clinical assessment device of claim 6, wherein at least one of said backrest and said seat is movable to tilt relative to said base.

8. The passive clinical assessment device of claim 6, wherein said plurality of sensors comprises at least one microphone supported on said backrest in position to register with the person’s back when seated on said seat.

9. The passive clinical assessment device of claim 6, wherein each of said pair of armrests supports a respective set of electrically-conductive contacts positioned to register with a region of the person’s wrists and hands as the person is seated on said seat, and wherein said base supports a third set of electrically-conductive contacts positioned to register with the person’s legs while the person is seated on said seat.

10. The passive clinical assessment device of claim 6, wherein at least one of said pair of armrests supports an inflatable cuff for automatedly obtaining a blood pressure measurement of a person seated on said seat.

11. The passive clinical assessment device of claim 6, wherein at least one of said pair of armrests supports a pulse oximeter clip for obtaining a measurement of a level of oxygenation of the person’s blood.

12. The passive clinical assessment device of claim 4, further comprising a scale operable to obtain a measurement of a weight of the person supported by the patient-supporting structure.

13. The passive clinical assessment device of claim 4, further comprising an electronic data management component operatively connected to at least one of said plurality of sensors to receive digital data acquired by said at least one of said plurality of sensors.

14. The passive clinical assessment device of claim 13, wherein said electronic data management component is operatively connected to said at least one of said plurality of sensors to receive at least one of breath sound data and heart sound data.

15. The passive clinical assessment device of claim 13, wherein said electronic data management component is configured to transmit data to an external computing device for data processing on said digital data.

16. The passive clinical assessment device of claim 13, wherein said electronic data management component is configured to perform data processing on said digital data.

17. The passive clinical assessment device of claim 16, wherein said electronic data management component is configured to perform data processing on said digital data to produce a 6-lead electrocardiogram (EKG).

18. The passive clinical assessment device of claim 1, wherein each of said plurality of sensors is configured for capturing biometric data relevant to a patient clinical assessment.

19. The passive clinical assessment device of claim 18, wherein at least one of said plurality of sensors is positioned to make physical contact with the person’s skin to acquire cutaneous signals.

20. The passive clinical assessment device of claim 19, wherein said plurality of sensors are positioned to make contact with skin on both upper extremities and at least one lower extremity of the person.

21. The passive clinical assessment device of claim 4, further comprising: a display pod supporting said caregiver display device in a position that is viewable from a vantage point opposite that of a person occupying the patient supporting device.

22. The passive clinical assessment device of claim 21, wherein said display pod further comprises a patient display device supported in a position to be viewable from a vantage point of the person occupying the patient supporting device.

23. The passive clinical assessment device of claim 21, wherein said display pod further comprises at least one touchless sensor positioned to obtain biometric data from the person occupying the patient supporting device.

24. The passive clinical assessment device of claim 23, wherein said at least one touchless sensor comprises an infrared (IR) temperature sensor capable of measuring temperature from a distance without direct contact with the person.

25. The passive clinical assessment device of claim 21, wherein said display pod further comprises a video camera positioned to capture images of the person occupying the patient supporting device.

26. The passive clinical assessment device of claim 21, wherein said display pod further comprises a tube, and wherein said passive clinical assessment device comprises at least one of hardware and software for performing an automated breath analysis on a breath sample received via said tube.

27. The passive clinical assessment device of claim 1, wherein said device further comprises a self-cleaning system comprising:
a reservoir configured to hold a cleaning solution;
a compressed gas source configured to hold compressed gas;
a dispenser structure in fluid communication with said reservoir and said compressed gas source and configured to dispense a mist of cleaning solution propelled by gas from said compressed gas source;
a valve operable to control a flow of cleaning solution and gas; and
a controller operable to selectively open and close said valve.

28. A passive clinical assessment device comprising:
a patient-engaging structure configured to register with a person’s anatomical structure in a predictable fashion, said patient-engaging structure comprising at least a base supporting a seat, and a backrest supported adjacent said seat;
a plurality of sensors supported on the patient-engaging structure in positions for registering with selected anatomical portions of a person while seated on said seat and against said backrest; and
an electronic data management component operatively connected to at least one of said plurality of sensors to receive digital data acquired by said at least one of said plurality of sensors.

29. The passive clinical assessment device of claim 28, wherein said electronic data management component is configured to transmit data to an external computing device for data processing on said digital data.

30. The passive clinical assessment device of claim 28, wherein said electronic data management component is configured to perform data processing on said digital data.

31. The passive clinical assessment device of claim 28, further comprising a scale operable to obtain a measurement of a weight of the person supported by the patient-supporting structure.

32. The passive clinical assessment device of claim 28, wherein said plurality of sensors comprises at least one microphone supported on said backrest in position to register with the person’s back when seated on said seat.

33. The passive clinical assessment device of claim 28, wherein each of said pair of armrests supports a respective set of electrically-conductive contacts positioned to register with a region of the person’s wrists and hands as the person is seated on said seat, and wherein said base supports a third set of electrically-conductive contacts positioned to register with the person’s legs while the person is seated on said seat.

34. The passive clinical assessment device of claim 28, further comprising an armrest supporting an inflatable cuff for automatedly obtaining a blood pressure measurement of a person seated on said seat.

35. The passive clinical assessment device of claim 28, further comprising an armrest supporting a pulse oximeter clip for obtaining a measurement of a level of oxygenation of the person’s blood.

36. The passive clinical assessment device of claim 28, further comprising: a display pod supporting a caregiver display device in a position that is viewable from a vantage point opposite that of a person occupying the patient supporting device.

37. The passive clinical assessment device of claim 28, wherein said display pod further comprises a patient display device supported in a position to be viewable from a vantage point of the person occupying the patient supporting device.

38. The passive clinical assessment device of claim 28, wherein said display pod further comprises at least one touchless sensor positioned to obtain biometric data from the person occupying the patient supporting device.

39. The passive clinical assessment device of claim 28, wherein said display pod further comprises a tube, and wherein said passive clinical assessment device comprises at least one of hardware and software for performing an automated breath analysis on a breath sample received via said tube.

40. A passive clinical assessment device comprising:
a patient-engaging structure configured to register with a person’s anatomical structure in a predictable fashion, said patient-engaging structure comprising at least a base supporting a seat, and a backrest supported adjacent said seat;
a plurality of sensors supported on the patient-engaging structure in positions for registering with selected anatomical portions of a person while seated on said seat and against said backrest; and
an electronic data management component operatively connected to at least one of said plurality of sensors to receive digital data acquired by said at least one of said plurality of sensors.

41. The passive clinical assessment device of claim 40, wherein each of said pair of armrests supports a respective set of electrically-conductive contacts positioned to register with a region of the person’s wrists and hands as the person is seated on said seat, and wherein said base supports a third set of electrically-conductive contacts positioned to register with the person’s legs while the person is seated on said seat.

42. The passive clinical assessment device of claim 41, wherein said plurality of sensors comprises at least one microphone supported on said backrest in position to register with the person’s back when seated on said seat.

43. The passive clinical assessment device of claim 42, further comprising:
at least one armrests; and
an inflatable cuff supported on said at least one armrest for automatedly obtaining a blood pressure measurement of a person seated on said seat.

44. The passive clinical assessment device of claim 43, further comprising a pulse oximeter clip supported on said at least one armrest for obtaining a measurement of a level of oxygenation of the person’s blood.

45. The passive clinical assessment device of claim 44, further comprising: a display pod supporting a caregiver display device in a position that is viewable from a vantage point opposite that of a person occupying the patient supporting device.

46. The passive clinical assessment device of claim 45, wherein said display pod further comprises a patient display device supported in a position to be viewable from a vantage point of the person occupying the patient supporting device.

47. The passive clinical assessment device of claim 46, wherein said display pod further comprises at least one touchless sensor positioned to obtain biometric data from the person occupying the patient supporting device.

48. The passive clinical assessment device of claim 47, wherein said display pod further comprises a tube, and wherein said passive clinical assessment device comprises at least one of hardware and software for performing an automated breath analysis on a breath sample received via said tube.
